(19) United States
(12) Patent Application Publication
(10) Pub. No.: US 2004/0111760 A1
(43) Pub. Date: Jun. 10, 2004

(54) Production of stilbenes in transgenic plants and the method of producing thereof

(76) Inventors: Tet Fatt Chia, Singapore (SG); Irene Ng, Singapore (SG)

Correspondence Address: Lawrence N Ginsberg, 21 San Antonio, Newport Beach, CA, 92660-9112, US

(21) Appl. No.: 10/432,152

(22) PCT Filed: Oct. 25, 2001
(86) PCT No.: PCT/SG01/00220

(30) Foreign Application Priority Data
Nov. 21, 2000 (SG) 0006741-3

(51) Int. Cl. A01H 1/00; C12N 15/82; A01H 5/00

(52) U.S. Cl. 800/278; 800/305

(57) ABSTRACT

A transgenic plant in which at least one stillbene synthase (STS) gene construct is transformed therein, and with the constitutive production of the corresponding stilbene synthesized by the transgenic STS enzyme, while maintaining normal physiological development The preferred embodiment contains transgenic resveratrol synthase (RS) transformed into a red plant. The method of production includes choosing a recipient plant that contains high levels of the precursors of the transgenic RS enzyme.

### FIELD OF INVENTION

This invention relates to transgenic plants and plant materials. In particular, the present invention is related to the production of resveratrol and other stilbenes in plants.

### BACKGROUND OF INVENTION

Cancer is the largest single cause of death in both men and women and chemo-prevention of cancer is one of the most direct ways to reduce morbidity and mortality. Cancer-preventive agents include nonsteroidal anti-inflammatory drugs, eg. indomethacin,aspirin, piroxicam and sulindac, all of which inhibit COX. In the search for new cancer preventive agents, over the past 30 years, thousands of plant samples and extracts were studied and hundreds of these extracts were evaluated on their potential to inhibit COX. In 1974, an extract from Cassia quinquangulata from Peru was identified as a potent inhibitor and the active ingredient was identified as resveratrol (3,5,4′-trihydroxy-trans-stilbene). In 1997, it was reported in Science Journal that resveratrol, a phytoalexin found in grapes and other foods was purified and shown to have cancer chemo-preventive activity in assays representing three major stages of carcinogenesis. Resveratrol was found to act as an antioxidant and anti-mutagen and to induce phase II drug-metabolizing enzymes (anti-initiation activity); it mediated anti-inflammatory effects and inhibited cyclooxygenase and hydroperoxidase functions (antipromotion activity) and it induced antiprogression activity of cancer. In addition, it inhibited the development of preneoplastic lesions in carcinogen-treated mouse mammary glands in culture and inhibited tumorigenesis in mouse skin cancer model (Jang M. S., Science. 275:218-220, 1997). These data and a host of other scientists around the world now strongly suggest that resveratrol, a common constituent in our diet merits investigation as a potential cancer chemopreventive agent in humans.

Alcohol, cardiovascular diseases and the French paradox has been hotly researched and pursuit by the medical scientific communities around the world for the past 20 years. Numerous studies over the years have shown that comparing alcohol intake and ischemic heart disease have shown either an inverse relation or a U-shaped curve in which the equivalent of 2 drinks per day of any kind of alcohol is associated with a decreased incidence of coronary disease compared with no drinks, while higher doses result in an increased risk of infarction and stroke. The cardio-protective effects of most alcoholic beverages are probably due to an elevation of high density lipoprotein and the ability of alcohol to prevent platelet aggregation and increase fibrinolysis; however, there is an increased favorable effect from red wine. The unique cardioprotective properties of red wine reside in the action of flavonoids and stilbenoids which are minimal in white wine (with the exception of champagne). The best researched flavonoids are resveratrol and quercetin, which confer antioxidant properties more potent than alpha-tocopherol. Grape juice has about half the amount of flavonoids by volume as red wine. Resveratrol, however, being a phytoalexin, is not normally produced in grapes unless it is attacked or infected by microbial pathogens.

As resveratrol is a phytoalexint, it is produced by at least 72 plant species spreading over 31 genera and 12 families. The best studied plants that produces resveratrol are grapes and peanuts. The US and especially German Universities have been actively looking at the plant-pathogen interaction in the 2 plants described Bayer AG, a giant chemical and pharmaceutical company have been actively sponsoring and working on this phytoalexin. They have isolated the genes (stilbene synthase) involved in resveratrol (phytoalexin) production and have shown that when expressed in transgenic plants, resveratrol can increase the resistance to pathogen attack on the plants. Bayer has also filed patents on the grape stilbene synthase gene that they have isolated. Fischer R (Plant J. 11(3):489-498, 1997) published a paper in The Plant Journal that over-expression of stilbene synthase gene in transgenic tobacco can lead to sterile pollen (due to the competition between chalcone synthase and stilbene synthase on the common precursor substrates).

Presently, there is also a company (PharmaScience) from Canada that is selling resveratrol in powder form and they claim that it is chemically synthesized. The Trade name is “Resverin”. They can only supply in small quantity at a very high price. Also, during the past to years, there were also many reports that showed that resveratrol is a phytoestrogen. Hence, resveratrol has also been implicated to mimic estrogen action and hence, may have a potential in non-steroidal estrogen supplements and also may prevent osteoporosis.

One difficulty of trying to tap the antioxidant and antimutagenic benefits of such stilbenes is that they are often phytoalexins and are therefore only found in infected or wounded plants, and not found in healthy plants, even if the gene is present naturally in the plant. Thus, although, for example, grapes have stilbene synthase (STS) genes and active STS enzymes, consumers typically do not benefit from consuming grapes, because resveratrol is not normally found in fresh, healthy grapes. There is therefore a need to produce plants that contain a high and constitutive level of one or more of the desired stilbenes.

### SUMMARY OF INVENTION

Accordingly, one aspect of the present invention is a transgenic plant in which at least one stilbene synthase (STS) gene construct is transformed therein, and with the constitutive production of the corresponding stilbene synthesized by the transgenic STS enzyme. In another aspect, fertility and physiological development of the transgenic plant may be controlled by selection of clones at specific ranges of expression of the stilbene. The plant is preferably a common vegetable that naturally produces high levels of the precursors for the transgenic STS in the edible portion. The plant is more preferably a leafy vegetable that is commonly eaten raw, but can be cooked if needed. The preferred STS gene is resveratrol synthase (RS). An example of plant that is a suitable recipient plant for the transformation of resveratrol synthase gene is the red-leaf lettuce (Lactuca Sativa). Red-leaf lettuce is also referred to as red lettuce. Other examples of recipient plants according to the present invention include colored vegetables and fruits, including, but not limited to, watermelon, strawberry, spinach, red cabbage, red sugarcane.

Thus, according to another aspect, the present invention is a transgenic plant in which at least one resveratrol synthase (RS) genes construct is transformed therein and with the constitutive production of resveratrol synthesized by the transgenic RS enzyme. The plant is preferably red-leaf lettuce.

According to yet another aspect, the present invention is related to an edible composition comprising portions of the transgenic plant, and one embodiment is a drink developed from and comprising the juice of such a transgenic plant. Thus, the preferred plant for this embodiment produces sufficiently large quantities of juices containing the transgenic resveratrol for the juice to be processed into a drink. In a further aspect of the present invention, dried vegetable and fruits containing transgenic resveratrol are provided. In this embodiment, the edible portion of the plant may contain any amount of fluid. In yet a further embodiment, plant extracts may be produced containing resveratrol from the transgenic plant. The extracts may be in a concentrated form or a unconcentrated form, such as in a powder form.

According to yet another aspect, the present invention is a method of producing a healthy transgenic plant containing a specific transgenic STS enzyme transformed therein. In the preferred embodiment, the transgenic plant obtained using a method according to the present invention further contains high and constitutive levels of transgenic resveratrol while maintaining normal physiological development. The method comprises (1) choosing a recipient plant containing high levels of the precursors of the transgenic STS enzyme; (2) providing a genetic vector comprising an STS gene or a portion of an STS gene encoding an STS enzyme, the STS gene or the portion of the STS gene being provided with a promoter suitable for constitutive expression of the STS enzyme in the recipient plant; (3) transforming the genetic vector into the recipient plant and (4) selecting and growing the transformed plant containing high and constitutive levels of the stilbene.

To check for endogenous stilbene synthase, oligonucleotides constructed according to conserved regions of a known STS gene may be used as a probe, followed by southern blot analysis of the genome of the candidate recipient plant.

For precursor level analysis, biochemical tests, such as HPLC, may be used. 4-Coumaroyl-CoA and malonyl-CoA are two precursors common to RS and other stilbene synthase enzymes. Alternatively, high precursors levels may be inferred from the level of other intermediates of the same biochemical pathway, such as the appearance of “redness” in the natural state of the plant. “Redness” is due to the accumulation of anthocyanins, intermediates of which are known precursor for RS.

The STS gene in the genetic vector may be a cDNA obtained from mRNA or genomic DNA isolated from plants containing the appropriate STS gene. Any plant that can synthesize the stilbenes of interest may be a candidate donor plant. The STS gene or cDNA of the STS gene of interest may be obtained using oligoprimers homologous to conserved region of known STS genes. The isolated STS DNA may be cloned into a conventional genetic vector with a conventional selectable marker (e.g. an antibiotic resistance gene) and a conventional promotor that can cause constitutive expression of the inserted STS DNA in the recipient plant.

In the specific preferred embodiment of this method, the genetic vector carries an RS gene or a portion of an RS gene encoding an RS enzyme resulting in high and constitutive levels of transgenic resveratrol being expressed in the transgenic plant. The precursors available for the transgenic RS enzyme are used naturally by the non-transgenic plant for anthocyanin production. The recipient plant is of a species that has a red colour, indicating high levels of naturally-occurring anthocyanins and its precursors.

In another preferred embodiment, the recipient plant may be regenerated by tissue culture methods, and the callus analyzed for precursors levels. Callus and plantlets that are found to express high and constitutive levels of the precursors of the transgenic resveratrol synthase enzyme are selected. This allows the transformed plants (that have begun to express the transgenic RS enzyme and thus begun to deplete precursors for the biosynthesis of the transgenic stilbene) to maintain good health during the course of tissue culturing, even though they grow into mature plants.

As used herein, STS genes refer to the family of genes that encode various STS enzymes. The STS enzymes catalyze the synthesis of different members of the stilbene family. Resveratrol synthase (RS) gene refers to a specific member of the STS gene family that encodes the reseveratrol synthase enzyme (RS enzyme). The RS enzyme catalyzes the conversion of 4-coumaroyl-CoA and malonyl-CoA to 3,4′,5-trihydroxy-trans-stilbene (resveratrol).

Redness is determined in a general manner and may be observable by the eye and generally accepted and well known by one in the art. Example of plants that are regarded as “red” include red-leaf lettuce (Lactuca sativa), red bayam (Amaranthus species), red cabbage (Brassica oleracea), red sugar beet (Beta vulgaris), purple cabbage, red-beet root, red amaranthus, red sugar cane, red spinach, red watermelon (Citrullus lanatus), red strawberry (Fragaria species), raspberry.

“Healthy” as used herein refers to a general state of health that is within the normal range of that species as observable according to appearance, such as size and colour.

“Fertile” as used herein refers to the ability of the species to form viable seeds.

### BRIEF DESCRIPTION OF DRAWINGS

FIG. 1 shows the last step in the biosynthetic pathway of resveratrol and naringenin chalcone.

FIGS. 2A-D are genetic maps of plasmid pBI 121 carrying the R1 RS gene (FIG. 2A), R65 RS gene (FIG. 2B), R14 RS gene (FIG. 2C) and R17 RS gene (FIG. 2D).

FIG. 3 is the Hydropathy curves of (a) Vitis vinifera cv. Optima RS (pSV21), (b) Arachis hypogaea RS (arqresol), (c) Pinus sylvestris STS (PSTS1) and (d) Phalenopsis sp. BS (pBibsy811).

FIGS. 4A-D show the 4 existing grape RS cDNA, pSV21, pSV25, pSV368 and VVLSTS as aligned using ClustralW. The primers used for isolating full-length Vitis vinifera cv. Red Flame RS genes are indicated in the boxed-in portion.

FIG. 5 are the restriction maps of (a) grape RS (pSV21, pSV25, pSV368 and VVLSTS) (b) grape RS introns (Vst1-1 and Vst2-1) (c) Vitis vinifera cv. Red Flame cDNA (R1, R5 and R8) (d) Vitis vinifera cv. Red Flame gDNA (G13, G14 and G17). Boxed-in regions are introns.

FIG. 6 is a DNA alignment between putative grape RS cDNA R6, gDNA G14 and grape RS VVLSTS.

FIG. 7 is a DNA alignment between putative RS cDNA R12, gDNA G13 and grape RS pSV21.

FIGS. 8A-E are HPLC elution profiles for the analysis of RV in the control (FIG. 8A) and the transgenic lines containing plasmids G14 (FIG. 8B), G17 (FIG. 8C), R1 (FIG. 8D) and R15 (FIG. 8E). The RV peaks are indicated by the arrows.

### DETAILED DESCRIPTION

The following example is used to illustrate the various aspects of the present invention.

Production of Resveratrol in Red-Leaf Lettuce

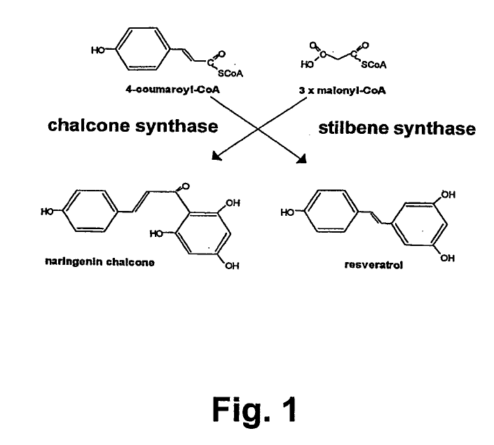

Resveratrol synthase (RS) gene from Vitis vinifera cv. Red Flame is the stilbene synthase gene used in this example as the member of the STS gene family for cloning and transformation. For ease of description and understanding, RV produced by the transgenic RS enzyme and stilbene produced by the transgenic STS enzymes are referred to as transgenic RV and transgenic stilbene respectively. Red leaf lettuce is chosen as the recipient plant for the RS gene in this example because it contains high levels of anthocyanin pigments for which 4-coumaroyl-CoA & malonyl-CoA are precursors. For example, naringenin chalcone is an intermediate of the anthocyanin biosynthetic pathway, in which 4-coumaroyl-CoA and malonyl-CoA are precursors. 4-coumaroyl-CoA and malonyl-CoA are also known precursors for resveratrol (RV), as shown in FIG. 1. Thus, there is plenty of precursors for the conversion to RV by RS within the transformed lettuce. Furthermore, the lettuce species was tested for the presence of RV-related genes in their natural genome by hybridizing with oligonucleotides that represent the homologous regions of various known RV genes. Southern blot anlaysis showed no hybridization occurring, indicating that there is no endogenous RS gene in lettuce making it a suitable recipient plant according to the preferred embodiment of the present invention. As a result, such that high and constitutive levels of RV may be attained in the transgenic red-leaf lettuce.

To start off, some red grapes (cultivar Red Flame) were obtained from the local supermarket. We UV the grapes for 10 minutes to induce the transcription of the resveratrol synthase transcript and waited for 12 hours and extraction mRNA from the UV irradiated grapes. We made oligoprimers 5′ and 3′ of the RS gene and through reverse transcription PCR, pulled out the cDNA sequence of the genes. We also extracted genomic DNA of the red flame grapes and again through PCR isolated the genomic RS genes (with 1 intron). The RS is a multi-gene family. Hence we mapped, sequenced and characterised all of them.

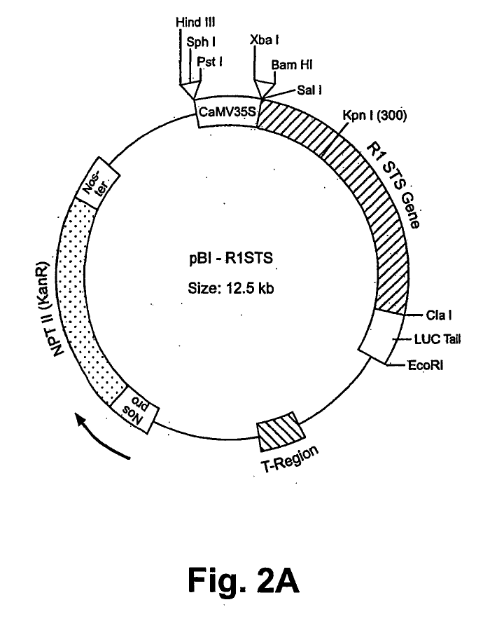

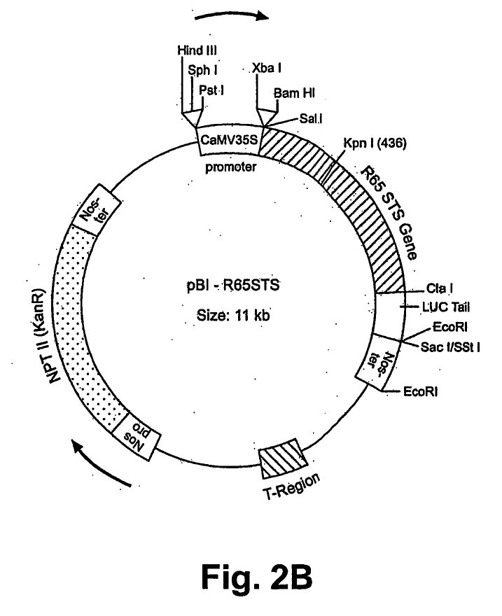

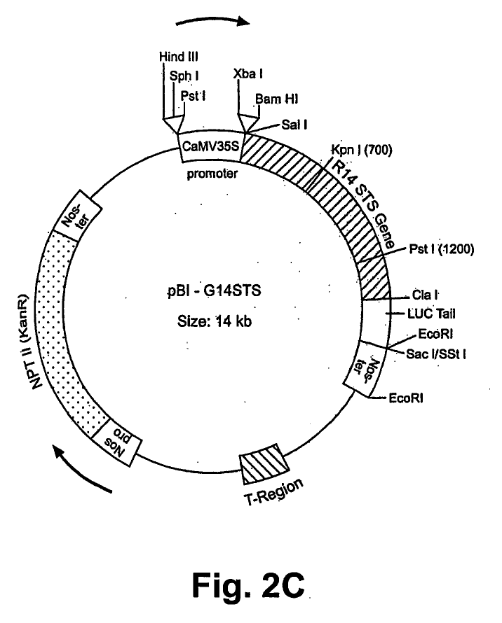

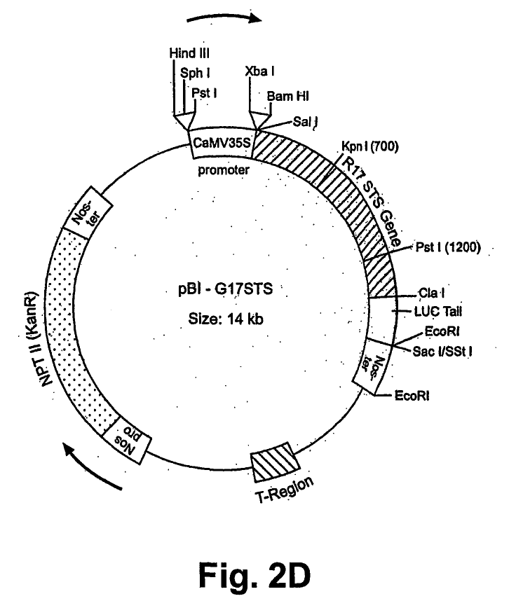

We chose 2 full length cDNA clones (named R1 and R65) and 2 genomic clones (named G14 and G17) and ligated them into an expression vector (pBI 121) driven by the Cauliflower Mosiac Virus (CaMv) 35S promoter. Clones R65, G14 and G17 were ligated into pBI 121 through the BamH1 and Sac1 restriction sites, while clone R1 was ligated into pBI 121 through the BamH1 and EcoR1 restriction sites. This plasmid expression vector was then transformed into Agrobacterium (strain LBA4404). The Agrobacterium that carries the pBI 121 with the RS gene is selected through Kanamycin resistance selection. Hence, we obtained 4 different Agrobacterium colonies carrying the 4 different constructs as shown in FIGS. 2A-D.

Concurrently, we also screened 5 different varieties of red-lettuce and assess their re-generation potential in tissue culture from cotyledon explants. The red-lettuce variety Red Salad Bowl was chosen because it showed the highest and fastest regeneration of plantlets produced from our tissue culture protocol.

The experiments were repeated, but this time the cotyledons were cut into squares of 2 mm sq. in area and were incubated for 30 minutes in the Agrobacteria. They were then rinsed and the tissue culture protocol that we established for Red-Salad Bowl was followed. Plantlets regenerated were selected using Kanamycin at 150 mg/L concentration in the shoot induction medium. These plantlets were rooted and planted out and grown for 30 to 35 days before the leaves were harvested for RNA, DNA and extraction for resveratrol quantification using HPLC.

Northern and Southern analyses showed that the RS genes (R1,R65, G14 and G17) were expressed. Organic solvent extraction of the leaves samples were done according to reported protocols for resveratrol analysis and were analysed using HPLC and a pure sample of resveratrol bought from Sigma chemicals was used as a standard reference.

The experiment using tobacco plants that had been similarly transformed, selected and was also done to serve as positive controls as a normal green plant.

The data from HPLC quantification show that the transgenic red-lettuce are capable of producing high and constitutive amount of resveratrol (over 4 ug/g fresh weight of leaf) as opposed to tobacco (best is around 0.36 ug/g f.w.). The transgenic red lettuce can produce up to 10 times the resveratrol as compared to transgenic tobacco when comparison is made using dry weight. Non transformed plants show no detectable resveratrol in them. A key observation and data obtained from our quantification of anthocyanin level of the transgenic red lettuce is that the anthocyanin level is reduced by half when compared to the non-transformed control. This data shows that some of the precursors, 4-coumaroyl-CoA and malonyl-CoA, are diverted to resveratrol production by RS and there is still potential in escalating the resveratrol concentration of the red-lettuce to a much higher level if we were to further over express the resveratrol synthase (RS) gene expression in the vegetable. Methods of over-expression include the use of stronger constitutive promotors or double promoters, use of the viral omega sequences for more efficient translation, and the use of other promoters like actin promoters.

This system also shows that it has the potential to be used in other coloured plants and fruits for high-level resveratrol yield. Assuming that we eat 100 g of vegetables daily, it will provide the resveratrol supply of >400 ug into our body daily. Hence, we foresee the potential of this novel invention in paving the way for a new generation of vegetable nutriceuticals that have chemo-preventive ability against Cancer, Cardio-vascular and other potential diseases.

The following are the detailed procedures used to obtain the transgenic red-leaf lettuce.

Choosing and Establishing Recipient Plant Material

4 varieties of red lettuces, namely, Lactuca sativa cv. Canasta, Lollo Rossa, Red Salad Bowl (Novartis seeds B.V, Holland) and Red Rapid (Known-you Seeds Co., Taiwan) were tested for their redness and re-generation ability in tissue culture.

The whole process of re-generation were done as described in Curtis et al., 1995, Methods in Molecular Biology, Vol. 44, Humana Press Inc. USA, pp.59-70, with some modifications. 10 seeds from each variety were surface sterilized using 10% Clorox for 10 minutes and rinsed three times with sterile R.O. water. The seeds were then sowed in 50 ml MS+B5 medium (Sigma Catalogue No.M-5519) in 250 ml conical flasks and grew at 23±2° C., 16 hours photoperiod, with light intensity of 18 μmol/s/m² (daylight fluorescent tubes) for 7 days.

The cotelydons of the 7-days old seedlings were excised, leaving the petiole intact but removed the apices of the cotelydons. Using a needle, the abaxial surface was poked repeatedly along the veins of the cotelydons. The cotelydons were floated on liquid UM medium (4.71 g/L MS salts and vitamins, 30 g/l sucrose, 2 g/l casein hydrolysate, 2 mg/l 2,4 dichlorophenoxyacetic acid (2,4-D, Sigma), 0.25 mg/l kinetin, 9.9 mg/l thiamine HCL, 9.5 mg/l pyridoxine-HCL, 4.5 mg/l nicotinic acid, 5.5 g/l phytagel, pH 5.8) for 10 minutes with their wounded surface in contact with the medium. The cotelydons were removed and immersed in UM agar medium. The explants were incubated for 2 days under the same conditions as for germinating seeds.

After 2 days in UM solid medium, the cotelydons were transferred to SI agar medium (4.71 g/L MS salts and vitamins, 30 g/l sucrose,0.04 mg/l NAA (Napthalene acetic acid) 0.5 mg/l Benzyl amino purine (BAP), 500 mg/l Carbeicillin, 100 mg/l cefotaxime, 150 mg/l kanamycin sulfate, 5.5 g/l phytagel, pH5.8) with the abaxial surface in contact with the SI agar medium. The cotelydons were incubated as for germinating seeds and sub-cultured to fresh SI agar medium every 21 days.

After 49 days, the explants that produced callus and shoots were transferred to 50 ml of SI agar medium with 0.11% (w/v) 2[N-morpholino]ethanesulfonic acid (MES).

Shoots that were approximately 1 cm high were transferred to 250 ml conical flasks each containing 50 ml of rooting agar medium (4.71 g/L MS salts and vitamins, 30 g/l sucrose, 0.04 mg/l NAA (Napthalene acetic acid), 150 mg/l kanamycin sulfate, 5.5 g/l phytagel, pH5.8). The shoots were incubated at the same conditions as germinating seeds.

Isolation of STS Genes from Grape and Construction of Genetic Vector

Plant Material

Mature fruits of commercially available grapevine Vitis cv. Red Flame were used as plant material for the isolation of grapevine RS genes.

Total RNA and Genomic DNA Extraction

0.2 g of Vitis cv. Red Flame skin tissues were ground in liquid nitrogen with the mortar and pestle. Both total RNA and gDNA were extracted using the same method described in Knapp and Chandlee (RNA/DNA Mini-Prep from a Single Sample of Orchid Tissue. Bio Techniques, 21:54-56), with some modifications. 2 ml of extraction buffer which contained 3% CTAB; 2% PVP; 1.42M NaCl; 20 mM EDTA, pH 8.0; 100 mM Tris, pH 8.0 and 5 mM ascorbic acid were used to extract total RNA or gDNA of Vitis. cv. Red Flame skin tissues. The samples were heated at 65° C. for 15 minutes, followed by a chloroform extraction to get rid of proteineous substances. ⅕ volume of 5% CTAB (5% CTAB and 0.7M NaCl) were added to the aqueous phase of the samples to remove polysaccharides and heated at 65° C. for 15 minutes. Another chloroform extraction was performed. 2 volume of ice-cold, 100% EtOH were added to the aqueous phase of the samples and incubated at −20° C. for 15 minutes. Total RNA and gDNA were pelleted after centrifuged for 15 minutes at 11,000 rpm, room temperature, using Eppendorf™ 5410C refrigerated centrifuge. The pellets were washed with 70% EtOH and dried in Eppendorf™ concentrator before dissolving in 50 μl TE (10 mM Tris-HCl, pH 8.0 and 1 mM EDTA, pH 8.0). 31 μl of the total RNA or gDNA isolated and 1 μl of loading buffer (0.025% bromophenol blue, 0.025% xylene cyanol, 30% glycerol in 1×TBE) were loaded onto 1% agarose gel together with 0.25 μg each of lambda DNA/HindIII and phiX174 DNA/HaeIII markers. Horizontal gel electrophoresis was run at 100V for ½ hour. The quantity and the quality of total RNA or gDNA extracted were visualized and calculated using EtBr stain and Stratagene's Eagle-Eye II Junior documentation system.

Primers Determination

All existing genes sequences of Vitis cv Optima STS (pSV21, pSV25 and pSV368), Vitis cv. Lambruscoa Foglia Frastagliata STS (VVLSTS), Phalenopsis sp. BS (pBibsy811 and pBibsy212), Arachis hypogaea (peanut) STS (arqresol and a00769) and Pinus sylvestris (Scots pine) STS (PSTS1 and PSTS2) were obtained from GenBank in the website of National Center for Biotechnology Information (NCBI). All homology searches were performed using ClustalW Multiple Sequence Alignment of BCM Search Launcher from Human Genome Center, Baylor College of Medicine, Houston Tex.

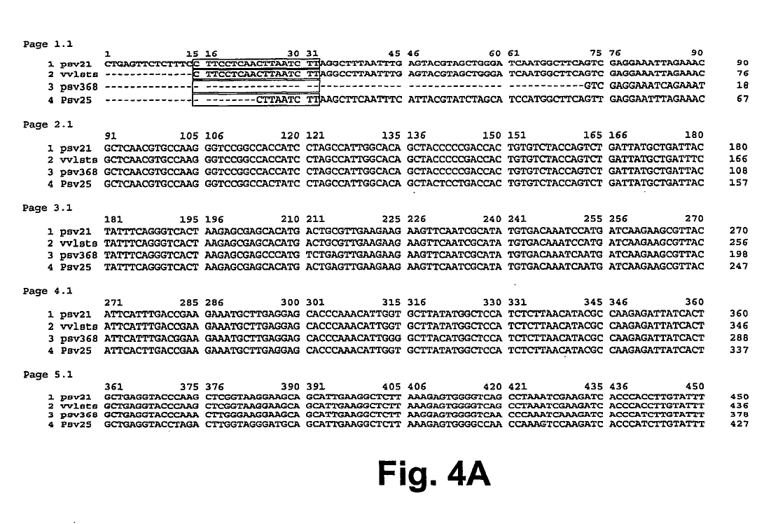

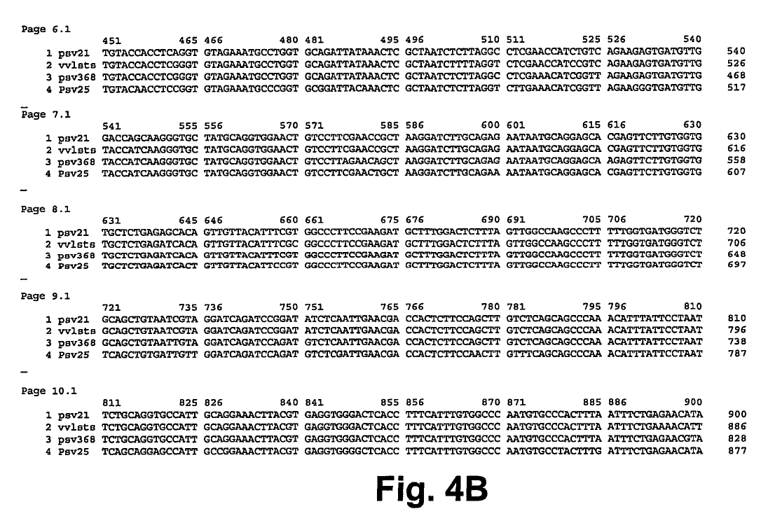

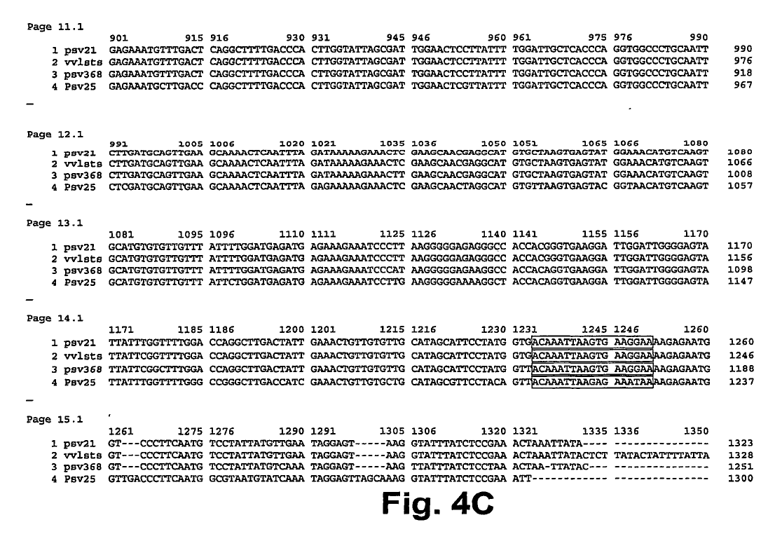

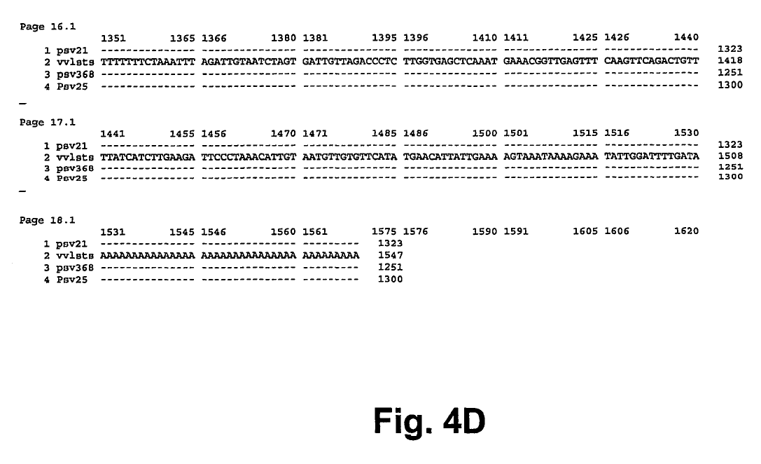

The primers used for isolation of full length grapevine RS genes were determined by multiple alignment of existing pSV21, pSV25, pSV368 (Melchior and Kindl, Optima. Arch. Biochem. and Biophy. 288:552-557, 1991) and VVLSTS (Spavoli F. Plant Mol. Biol. 24:743-755, 1994) as indicated in FIGS. 4A-D.

After determination of the primers sequences and the melting temperatures of the primers, they were custom synthesized commercially by Gibco BRL Custom Oligonucleotide Synthesis Service, L.T.I., U.S.A.

Reverse Transcription of Total RNA

1 μg of UV-induced Vitis cv. Red Flame skin total RNA leaves total RNA was used as templates to allow the annealing of the 3′ primer—35GSTS2a at 65° C. for ten minutes. After the primer was annealed, the total RNA was reverse transcribed with 200 units/μl Superscript™II reverse transcriptase (Gibco BRL, LTI, U.S.A), SuperscriptII™ 1× reaction buffer, 10 mM DTT and 200 μM dNTP in the final volume of 50 μl. The reverse transciption was carried out in Perkin Elmer GeneAmp PCR system 2400 at 42° C. for 1 hour, then Superscript™II reverse transcriptase was inactivated at 70° C. for 15 minutes.

The cDNA was purified through Tris-buffered phenol and chloroform:isoamyl alcohol (24:1) extraction. The aqueous phase was then precipitated with 1/10 volume of 3M sodium acetate and 2.5 volume of ice-cold, 100% EtOH. The pellet was dissolved in 20 μl of sterile nmlli-Q water.

Polymerase Chain Reaction of cDNA and gDNA

Both 20 μl of cDNA and 20 ng of gDNA of Vitis cv. Red Flame skin, were amplified by Polymerase Chain Reaction (PCR) in Perkin Elmer GeneAmp PCR system 2400. The PCR reaction at a total volume of 50 μl included 5 units/μl Thermus flavus (Tfl) DNA polymerase in 1×Tfl reaction buffer provided (Promega, U.S.A), 200 ng/μl each of 5′ primer 35GSTS1 and 3′ primer 35GSTS2a, 200 μM dNTP, 1.5 mM magnesium sulfate and topped up with sterile milli-Q water.

PCR amplification for grapevine RS genes was done by holding at 92° C. for 5 minutes, followed by 40 cycles of denaturing time of 1 minute at 92° C., annealing at 55° C. for 2 minutes and an extension time of 2 minutes at 72° C. Further extension at 72° C. for 6 minutes completed the PCR.

5 μl of PCR reaction were separated and quantified by Horizontal gel electrophoresis in 1% agarose. After determining the presence of the desired MW fragments, the rest of 45 μl PCR reaction were selective precipitated with 1/10 volume of 10× STE (10 mM Tris.Cl, pH 8.0; 100 mM NaCl and 1 mM EDTA, pH 8.0), 1/10 volume of 4M NH₄OAc and 2.5 volume of ice-cold, 100% EtOH. The pellets were dissolved in TE for ligation into pGEM-T (+) vector.

Cloning into pGEM-T(+)

The cDNA and gDNA PCR products of Vitis cv. Red Flame skin were cloned into pGEM-T(+) vector using the pGEM-T(+)™ vector system kit (Promega, U.S.A). The cDNA and gDNA PCR products in the ratio 1:3 (vector:insert) were ligated into pGEM-T(+) vector using 3 units/μl of T4 DNA ligase, T4 DNA ligase 1× buffer in the total volume of 10 μl and incubated at 16° C. overnight.

After ligating, 10 μl of the ligation reaction mix were transformed into 200 μl of XL1-Blue competent cells (Stratagene, U.S.A). They were put in ice for 10 minutes, followed by 5 minutes at 37° C., then back in ice for 1 minute. 1 ml of plain LB broth was added and incubated at 37° C. for 1 hour. After 1 hour recovery time, the cells were collected through centrifugation at 11,000 rpm for 30 seconds, then resuspended in 50 μl plain LB broth. 50 μl were used to spread onto 1.5% LB agar plates with 100μg/ml ampicillin. These plates were incubated at 37° C. overnight.

The positive clones with the correct size inserts were selected using restriction enzymes SalI and ClaI (NEB Biolabs, U.S.A) digest following the manufacturer's recommended conditions after plasmid miniprep.

Characterization of Putative Vitis cv. Red Flame STS Genes

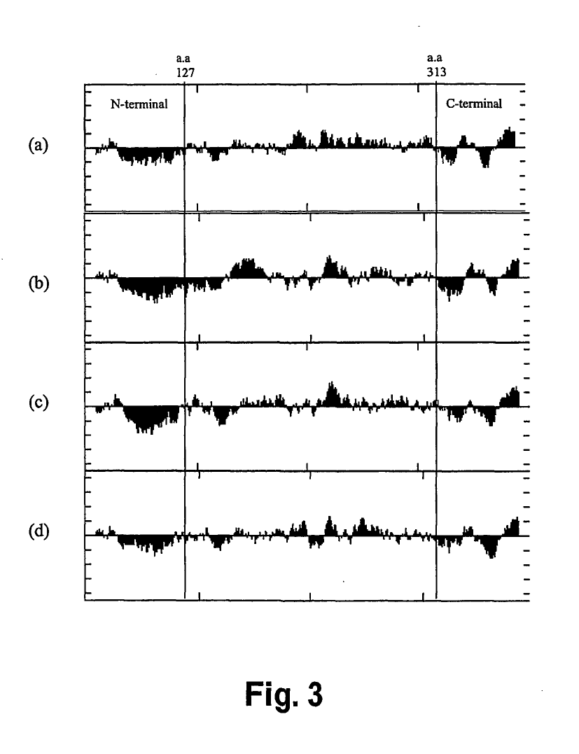

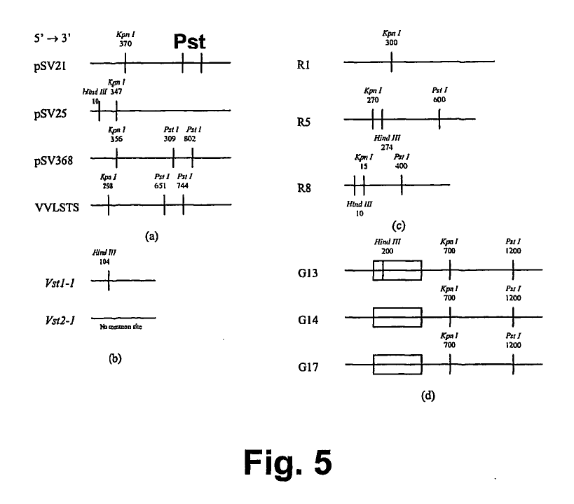

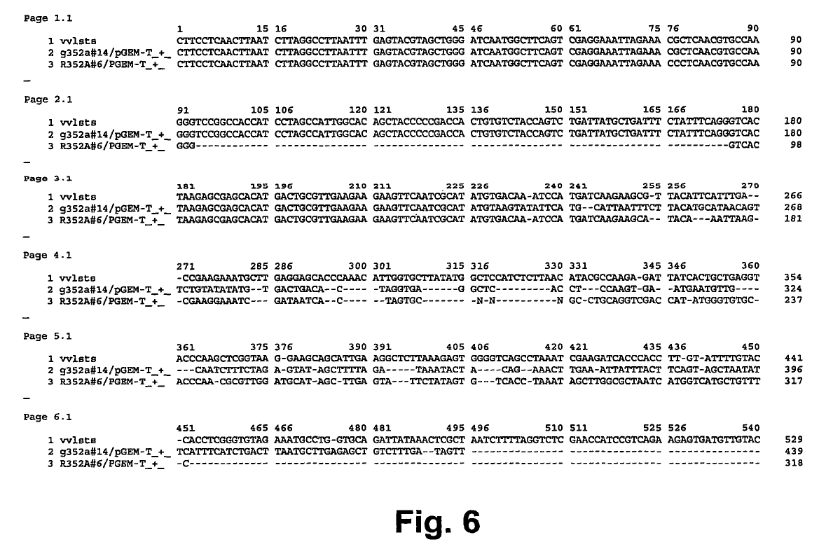

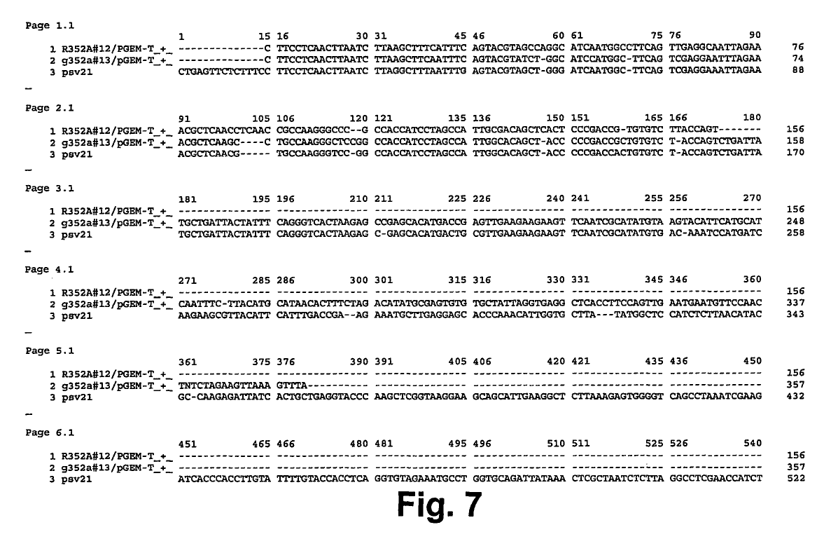

After the putative Vitis cv. Red Flame RS genes had been isolated, they were characterized by restriction enzyme mapping (FIG. 5), sequence analysis (FIGS. 6 and 7) and plotting of hydropathy curves (FIG. 3).

Restriction Enzyme Mapping

Restriction enzyme mapping of the existing grapevine RS genes (pSV21, pSV25, pSV368 and VVLSTS) was identified using the website Webcutter 2.0.

According to the restriction enzyme maps obtained, PstI, KpnI, and HindIII (NEB, U.S.A.) were used to digest the putative clones of Vitis cv. Red Flame RS genes at the manufacturer's recommended conditions. After digestion, the reactions were analyzed on 1% agarose gel using Horizontal gel electrophoresis running at 100V for 45 minutes. Gels were viewed using Eagle-Eye II Junior documentation system (Stratagene, U.S.A.)

Sequence Analysis

The sequences of the putative Vitis cv Red Flame RS genes were analyzed using dideoxy nucleotide chain termination method. Sequenase™ Version 2 sequencing kit (Amersham-Pharmacia, Sweden) was used for the sequencing reaction with forward primer (ssDNA sequencing) and reverse primer (dsDNA sequencing). The reactions were labeled using 35S-dATP (NEN, U.S.A.). A 6% polyacrylamide gel was ran at 50 W using the Sequencing Apparatus S2 (L.T.I., Inc., U.S.A.). Autoradiography was performed by exposing to Kodax MR Bio-Max film in a Kodax intensified screen cassette for approximately 16 hours. The films were then developed with Kodax Developer and Kodax Fixer. The sequences were read manually. The first 300 bases of the 5′ sequences of clone R65 showed that it belongs to the PSV21 group of grape RS gene.

Hydropathy Curves

Hydropathy curves were plotted for Vitis cv. Optima RS (pSV21), Phalenopsis sp. BS (pBibsy811), Arachis hypogaea RS (arqresol) and Pinus sylvestris STS (PSTS1) using the hydropathy plot website maintained by Biochemistry and Molecular Biology at Pennsylvania State University. The hydropathy curves were plotted based on Kyte and Dolittle method.

Cloning Vitis cv Red Flame RS Genes Into Expression Vector

4 clones of Vitis cv. Red Flame RS genes were cloned into expression vector pBI 121. Clones R65, G14 and G17 were cloned into pBI 121 vector through the BamH1 and Sac1 restriction sites. Clone R1 was cloned into pBI 121 through the BamH1 and EcoR1 restriction sites respectively. The genes were driven by a constitutive CaMV 35S RNA promoter. The cloned vectors are shown in FIGS. 2A-2D.

Transformation of Plasmid Containing RS Gene Constructs Into Agrobacterium LBA4404

1. Prepare sufficient YEP for liquid culture and plating and restreak of transformants. Requires 5 ml liquid culture per transformation, 1 ml for outgrowth, 20-40 ml for plates. (YEP: 10 g Bacto-peptone, 10 g Bacto-yeast extracts, 5 g NaCl; for solid 10 g/l phytagar).

2. Grow Agrobacteria LBA 4404 colony O/N in 2 ml YEP at 28° C.

3. Add to 50 ml of YEP in reserved 250 ml flask and shake at 250 rpm to OD of 0.5 to 1 at wavelength of 600 nm.

4. Chill culture on ice for 5 min. Spin ˜5,000 rpm for 5 min.

5. Carefully decant supernatant and resuspend tube in 1 ml of 0° C., 20 mM CaCl₂. Resuspend gently and strictly at 0° C.

6. Dispense 0.1 ml aliquots into prechilled microfuge tubes.

7. Add 1 ug of DNA to cells, mix gently but thoroughly, then freeze in dry ice-EtOH bath.

8. Place cells in 37° C. bath, 5 min.

9. Add 1 ml YEP and incubate with gentl shaking 2-4 hr.

10. Centrifuge for 45 sec at 4000 rpm in a microfuge. With pipet tip discard all but 100-200 ul of medium, resuspend cells in remaining medium by pipeting and/or vortexing and plate on 25 ug/ml Kanamycin. Incubate at 28° C. Colonies should appear in 2-3 days.

Transformation of Red Lettuce by Agrobacterium

1. Lettuce seeds, Var Red Salad Bowl were surface sterilised with 10% w/v chlorox for 10 minutes and rinsed 3 times with clean distilled water.

2. Seeds were than germinated in germination medium contained in 9 cm diameter Petri dishes (30 seeds/dish). Incubated at 24° C., 16 hr light at 18 umol/s/m² intensity.

3. Agrobacterium tumefaciens strain LBA4404 containing the binary vector pBI121 constructs (R1, R65, G14 and G17) were grown in LB medium with pH 7 and Kanamycin sulfate at 50 mg/L and 2 mg/L tetracycline-HCL for 1 day in a shaker at 210 rpm.

4. Pour 20 ml aliquots of UM agar medium into 9 cm diameter Petri dishes and allow to solidify.

5. Soak one sterile 7 cm diameter Whatman filter paper in liquid UM and place onto the surface of the UM agar medium.

6. Excise the cotyledons from 7 day old seedlings, leaving the petiole intact, but remove the apices of the cotyledons. Score the abaxial side using a fine needle. Using a scalpel, make shallow cuts (1 mm apart) transversely across the surface of the cotyledons. Float the cotyledons for 10 mins in an Agrobacterium liquid culture. Controls are done similarly except Agrobacteria is not used.

7. Remove the cotyledons and blot dry with sterile filter paper and transfer to the prepared UM dishes (10 cotyledons per dish). Incubate for 2 days under the same conditions as for germinating seeds.

8. Set up test plates as follow:

9. A. Control explants without Agrobacterium inoculation on:

i. SI medium

ii. SI medium+100 mg/L kanamycin sulfate;

iii. SI medium+150 mg/L Kanamycin sulfate

B. Explants inoculated with Agrobacterium on:

i. SI medium+100 mg/L Kanamycin sulfate and:

ii. SI medium+150 mg/L kanamycin sulfate.

10. Transfer the explants to SI medium, submerging the petiole ends of the cotyledons into the medium to a depth of about 2 mm. Incubate as for germination of seeds and subculture to fresh SI agar medium every 17 days.

11. After 40 days, transfer those explants that have produced callus and shoots to a 250 ml capacity flasks, each containing 60 ml of SI agar medium with 0.11% w/v Carbeicillin. 4 explant per flask, incubate at high light intensity of 80 umol/s/m².

12. Transfer shoots when approx. 1 cm high to the rooting medium, incubate at high light intensity of 80 umol/s/m².

13. When rooted, carefully remove plants from the containers, wash away the agar and transfer the plants into 10 inch pots filled with vermiculite. Enclose the plants in clear polyethylene bags for 3 days and remove them. The plants are then grown under full sunlight and fertilised with Graviota fertilizer. After 35 days, these plants were assayed for Northern, Southern and also extracted using organic solvent for HPLC analyses for resveratrol yield.

Selection of Transgenic Plants

After piercing, the cotelydons with Agrobacteria carrying the constructs. The cotelydons were then immersed in UM agar medium (˜15 cotelydons/plate) and incubated at 23±2° C., 16 hours photoperiod, with light intensity of 18 μmol/s/m² (daylight fluorescent tubes) for 2 days.

After 2 days, the cotelydons were placed on SI agar medium with abaxial surfaces in contact with the medium. The cotelydons were grown in the conditions mentioned above. For the color selection, the cotelydons were sub-cultured into fresh SI agar medium every 21 days. Small shoots of ˜1 mm and that were red in color were discarded while the pink and green plantlets were placed in UM agar medium for 2 days. Pink or green plantlets that turned red at this stage were also discarded, while those remained pink or green, were subcultured into fresh SI agar medium. Those plants that were ˜1 cm in height were placed into rooting agar medium.

For kanamycin selection, after placing in SI agar medium for a week, the cotelydons were transferred to SI+150 μg/ml kanamycin sulfate agar medium. Subculturing was done every 4 weeks to SI+150 μg/ml kanamycin sulfate agar medium.

Using the procedures described above, Red Flame RS genes were successfully cloned into Red-lettuce and transgenic resveratrol produced in the transgenic plants. The results obtained by using the methods described above are shown below:

Recipient Plant Material

The 4 cultivars Lactuca sativa were tested for the anthocyanin level and re-generation ability in vitro. The amount of precursors for resveratrol (i.e. 4-coumaroyl-CoA and malonyl-CoA) can then be inferred from the anthocyanin levels, since 4-coumaroyl-CoA and malonyl-CoA are common precursors for these two biosynthetic pathways. It is understood that the levels of 4-coumaroyl-CoA and malonyl-CoA may be determined directly by one skilled in the art, and is considered within the scope of the present invention.

Table 1 shows analysis of redness of cotyledons after 2 days in UM medium, color of calli after 14 days in SI medium and the re-generation ability after 37 days in SI medium for Lactuca sativa cv. Canasta, Lollo Rossa, Red Rapid and Red Salad Bowl.

From Table 1 as shown below, the cotyledon of Lactuca sativa cv. Red Salad Bowl was shown to be the most red in color and the Lactuca sativa cv. Lollo Rossa cotyledon was the least red, after 2 days in UM medium. Calli were formed after 2 weeks in SI medium showed that Lactuca sativa cv. Canasta, Lollo Rossa and Red Rapid had more light green calli than other colors calli. As for the Lactuca sativa cv. Red Salad Bowl, red calli was in higher percentage than other colors calli. Only Lactuca saliva cv. Red Rapid and Red Salad Bowl had calli of pink, dirty-red and white colors.

TABLE 1

| Lactuca | Redness | Calli | | | | | | Regene- |
|---|---|---|---|---|---|---|---|---|
| sativa | of coty- | Light | Dark | | | Dirty | | ration |
| cv. | ledons | green | green | Red | Pink | red | White | ability |
| Canasta | ++ | +++ | + | ± | − | − | − | + |
| Lollo | + | +++ | ± | + | − | − | − | ++ |
| Rossa | | | | | | | | |
| Red | +++ | +++ | ± | ++ | + | ± | ± | +++ |
| Rapid | | | | | | | | |
| Red | ++++ | + | + | +++ | + | + | + | ++++ |
| Salad | | | | + | | | | |
| Bowl | | | | | | | | |

Legends: ± means <30%; + means 30%-50%; ++ means 50%-70%; +++ means 70%-90%; ++++ means >90%

The 2,4-D (2,4-dichlorophenoxyacetic acid), an auxin, triggers the formation of anthocyanins. High anthocyanins production is one of the criteria for choosing the cultivar to be used for transformation. The higher the anthocyanins level, that means there are more substrates available (4-courmaroyl-CoA and malonyl-CoA). Therefore, when the STS genes were transformed into the red lettuce, the gene product had ample substrates to use. This facilitated the color selection of transgenic plants, as the color change to light pink or dark pink will be more prominent if the untransformed portions of the cotyledons are red. Furthermore, the cotyledons of Lactuca saliva cv. Red Salad Bowl has a uniform color throughout the cotyledons (FIG. 3a).

As for the re-generation ability, more than 90% of Lactuca sativa cv. Red Salad Bowl calli re-generated into plantlets. Lactuca saliva cv. Red Salad Bowl had the highest re-generation ability compared to Lactuca saliva cv. Canasta, Lollo Rossa and Red Rapid. Lactuca sativa cv. Red Rapid had approximately 70% to 90% calli re-generated into plantlets. This will shorten the time needed to select transgenic plants. Hence, Lactuca saliva cv. Red Salad Bowl is chosen as the plant materials for transformation of Vitis vinifera cv. Red Flame RS genes.

RS Genes from Red Flame Grape

Primers Determination

ClustalW alignment of existing grape RS cDNA isolated from Vitis vinifera cv. Optima (pSV21, pSV25 and pSV368) and Vitis vinifera cv. Lambruscoa Foglia Frastagliata (VVLSTS) showed that they were quite similar and shared high homology of 87.5% (FIG. 4). Consensus regions at the 5′ and 3′ ends of the sequences were determined for isolating the full-length genes from Vitis vinifera cv. Red Flame. 5′ primer determined was 5′ GTC GAC CTT CCT CAA CTT AAT CTT 3′ (designated as 35GSTS1) and 3′ primer was 5′ ATC GAT TTC CTT CAC TTA ATT TGT 3′ (designated as 35GSTS2a). They were highlighted in red in FIG. 4. 35GSTS1 contained a SalI linker while 35GSTS2a contained ClaI linker, both at the 5′ ends.

cDNA and gDNA Putative Clones of Vitis vinifera cv. Red Flame RS Genes

Vitis vinifera cv. Red Flame cDNA of MW 1.3 kb was obtained after RT and PCR. Out of 18 clones in pGEM-T(+), 12 clones contained insert sizes ranging from 0.9 kb to 1.6 kb after digesting with SalI and ClaI. While for the gDNA, 1.6 kb fragment was obtained. All the 18 clones that were digested with SalI and ClaI contained insert sizes of 1.5 kb to 1.6 kb.

Restriction Enzyme Mapping

12 cDNA clones and 18 gDNA clones of Vitis vinifera cv. Red Flame were subjected to PstI, KpnI and HindIII digestion. 3 clones of cDNA of Vitis vinifera cv. Red Flame were found to have similar restriction enzyme maps as the existing grape RS genes (FIG. 5a). The restriction mappings of the cDNA 3 clones, R1, R5 and R8 were shown in FIG. 5c. The size of R1 was 1.3 kb and it possessed 1 KpnI site and none of the PstI and HindIII sites. As for R5, it had 1.2 kb size and 1 site each for PstI, KpnI and HindIII sites (Table 2). These 3 clones together with 3 other clones (R3, R6, R12), whose restriction enzyme maps did not show any similarity to that of the existing grape RS, were subjected to sequence analysis.

The MW of the inserts of the gDNA clones of Vitis vinifera cv. Red Flame was listed in Table 2. Out of 18 clones, 4 clones were of size 1.5 kb and 5 clones were of size 1.6 kb. All clones had the PstI and KpnI sites, while only 1 clone (G13) had the HindIII site. The restriction maps for G13, G14 and G17 were shown in FIG. 5d and they were similar to each other. All of the 9 clones were subjected to sequence analysis.

TABLE 2
The MW (in kb) and the presence of PstI, KpnI and HindIII
in Vitis vinifera cv. Red Flame cDNA and gDNA restriction
maps. Legend: ✓ - site present, X - site absent.

| Putative clones | Size (kb) | Pst I | Kpn I | Hind III |
|---|---|---|---|---|
| cDNA | | | | |
| R1 | 1.3 | X | ✓ | X |
| R5 | 1.2 | ✓ | ✓ | ✓ |
| R8 | 0.9 | ✓ | ✓ | ✓ |
| gDNA | | | | |
| G4 | 1.5 | ✓ | ✓ | X |
| G5 | 1.5 | ✓ | ✓ | X |
| G9 | 1.5 | ✓ | ✓ | X |
| G11 | 1.5 | ✓ | ✓ | X |
| G13 | 1.6 | ✓ | ✓ | ✓ |
| G14 | 1.6 | ✓ | ✓ | X |
| G15 | 1.6 | ✓ | ✓ | X |
| G16 | 1.6 | ✓ | ✓ | X |
| G17 | 1.6 | ✓ | ✓ | X |

Sequence Analysis

First 300 bp sequence analysis and using BLAST program (website: http://www.ncbi.nlm.nih.gov/BLAST/), showed that the cDNA clones, R1 and R5 were 94.5% homology to pSV25 as shown in Table 3. But R5 had 85 bp missing. For R3, R65 and R12, they were of the same sequence and shared 97.5% homology to pSV21. R6 was homologous to VVLSTS with 99% homology level but 82 bp were missing as shown in FIG. 6.

As for the gDNA clones of Vitis vinifera cv. Red Flame, G13 revealed 93% homology to pSV21 (Table 3). 4 gDNA clones (G5, G9, G14 and G17) as shown in Table 3, was 99% homology to VVLSTS. After the first 300 bp sequencing analysis, these 4 gDNA clones were found to be of the same sequence.

TABLE 3
Homology level of Vitis vinifera cv. Red Flame cDNA and
gDNA STS putative clones with existing grape RS genes
(pSV21, pSV25, pSV368 and VVLSTS)

| | | |
|---|---|---|
| Putative clone | | |
| Existing | cDNA | gDNA |

| STS genes | Clones | % | Clones | % |
|---|---|---|---|---|
| pSV21 | R3, R12 | 97.5 | G13 | 93 |
| pSV25 | R1, R5 | 94.5 | — | — |
| pSV368 | — | — | — | — |
| VVLSTS | R6 | 99 | G5, G9, G14, | 99 |
| | | | G17 | |

Since clones that were homologous to pSV21 and VVLSTS were isolated from both cDNA and gDNA, alignment was done between these clones. In FIG. 7, R12 and G13 were similar but not identical to each other. Same result was obtained for R6 and G14 (FIG. 6).

Hydropathy Curves

The hydropathy curves for Vitis vinifera cv. Optima RS (pSV21), Phalenopsis sp. BS (pBibsy811), Arachis hypogaea RS (arqresol) and Pinus sylvestris STS (PSTS1) shown in FIG. 6 were similar to each other. They were divided into 3 main domains. The hydrophobic N-terminal (a.a. 1 to 127), hydrophilic middle portion (a.a. 128 to 313) and a mixture of hydrophobic and and hydrophilic C-terminal (a.a. 314 to 392). The a.a position is based on pSV21.

Clones pUCSTS-R1, R3 and R12 are full-length cDNA STS genes from Vitis vinifera cv. Red Flame. According to the sequence analysis, they are homology to pSV25 (R1) and pSV21 (R3,and R12). The MW of these cDNA clones do not correspond to the expected MW, which is in the range of 1.179 kb to 1.237 kb, when the 5′ (35GSTS1) and 3′ (35GSTS2a) primers are used. But, they correspond to the MW of pSV21, pSV25, pSV368 and VVLSTS, which are 1.323 kb, 1.3 kb, 1.251 kb and 1.547 kb respectively (Melchior and Kindl, Optima. Arch. Biochem. and Biophy. 288:552-557, 1991 and Spavoli F., Plant Mol. Biol. 24:743-755, 1994).

Furthermore, the restriction maps of R1, R3 and R12 are not the same as that of the existing grape RS genes (FIG. 5a and FIG. 5c). These differences can be explained by the different cultivars used.

As for pUCSTS-R5 and R6, sequence analysis revealed that they have 85 bp and 82 bp deletion respectively. Although R5 is homologous to pSV25 and R6 is homologous to VVLSTS, this deletion causes a shift in the open reading frame. As translation uses codon of threes to make amino acids, a shift in the open reading frame, will affect the functionality of the proteins produced. Therefore, these 2 clones are considered as cloning artifacts.

gDNA clones pUCSTS-G5, G9, G13, G14 and G17 isolated are full-length STS genes from Vitis vinifera cv. Red Flame. The sizes of the clones which are in the range of 1.5 kb to 1.6 kb, correspond to the gDNA STS genes Vst1 and Vst2 isolated from Vitis vinifera cv. Optima (Wiese W., Plant Mol. Biol. 26:667-677, 1994).

Due to the sequences of Vst1 and Vst2 are not available except for the sequences of the introns, sequence analysis of gDNA STS clones cannot be compared to Vst1 and Vst2. But, Vst1 is 98% homology to pSV25 (Wiese W., Plant Mol. Biol. 26:667-677, 1994). Therefore, the gDNA STS clones can also be compared to pSV25. From the sequence analysis, the gDNA clones obtained are homologous to either pSV21 (G13) or VVLSTS (G5, G9, G14 and G17). This again can be explained by the different cultivars used. Another reason maybe the genes that are similar to pSV25 are not being isolated in this experiment.

Restriction maps of the gDNA RS clones from Vitis vinifera cv. Red Flame do not show any similarity to those of the existing RS genes (FIGS. 5a, 5b and 5d). Different cultivars used maybe the reason for this result.

Due to the full sequences have not been sequenced for cDNA and gDNA RS genes of Vitis vinifera cv. Red Flame, the hydropathy curves for these clones of RS genes cannot be plotted. However, the hydropathy curves for Vitis vinifera cv. Optima STS (pSV21), Phalenopsis sp. BS (pBibsy811), Arachis hypogaea STS (arqresol) and Pinus sylvestris STS (PSTS1), as shown in FIG. 3, were similar to each other with 3 main domains. Preisig-Müller R. Biochem. 36:8349-8358, 1997. showed that the N-terminals of STS and BS were responsible for the substrate recognition or specificity, while the C-terminals were responsible for the product formation. STS(s) of different plants are quite conserved. Also, despite Vitis vinifera cv. Optima (pSv21) and Arachis hypogaea RS (arqresol) produced resveratrol, while Pinus sylvestris STS (PSTS1) produced pinosylvin as product (Schanz S., FEBS. 313(1):71-74, 1992), the STS(s) between these 3 plants are similar.

STS and BS are conserved too. STS utilizes 4-courmaroyl-CoA and malonyl-CoA whereas BS utilizes m-hydrophenylpropionyl-CoA and malonyl-CoA, despite this, their hydropathy curves are similar. According to Fliegmann J. (Plant Mol. Biol. 18:489-503, 1992), STS can utilizes substrates other than their originally preferred ones, but in a lower rate (that is Km value is lower). This may provide the explanation of the similar hydropathy curves.

Analysis of Transformed Plants

Plants transformed with the various gene constructs were analyzed for RV concentrations. Results are shown in Table 4.

TABLE 4

Estimated resveratrol concentration (μg/g f.w.)

| STS construct | Nicotiana tabacum | Lactuca sativa red lettuce |
|---|---|---|
| G14 | 0.09 | 0.60 |
| G17 | 0.27 | 4.80 |
| R1 | 0.15 | 0.94 |
| R65 | 0.36 | 0.40 |
| Ctrl | 0.00 | 0.00 |

Anthocyanin levels were also analyzed. Table 5 shows: anthocyanins level expressed as A₅₃₀/g for control and transgenic L. sativa cv Red Salad Bowl.

TABLE 5

| Sample | A₅₃₀/g |
|---|---|
| PBI (control) | 0.0102 |
| G14 | 0.0133 |
| G17 | 0.0048 |
| R1 | 0.0133 |
| R65 | 0.0047 |

Analysis showed a significant reduction of anthocyanins levels when the plants were planted under full sunlight and observed visually. Hence, anthocynanin levels seen in the transgenic lettuce is inversely proportional to the resveratrol yield.

Seeds are not viable in red lettuce plants that contained high levels (>3 ug/g.f.w.) of resveratrol. At a lower RV level (<1.5 ug/g.f.w.), the seeds are viable. The juice of these transformed red lettuce plants can produce juice with RV concentration of approximately 1 ug/ml (by obtaining undiluted juice of transformed plants expressing approximately 1.2 ug RV per g.f.w.) to approximately 4 ug/ml (by obtaining undiluted juice of transformed planted expressing approximately 4.8 ug RV per g.f.w.) The juice may be consumed directly, and the RV absorbed by the consumer, since RV expressed naturally in plants is known to be glycosylated and easily absorbed by the body. Alternatively, the transformed plants may be consumed as dried fruit or vegetable, such that a higher amount of RV can be consumed in each serving.

Stability of the Gene

Regeneration from seeds of the transgenic plants with <1.5 ug/g.f.w. of RV expression shows that the transgene is stable for at least 2 generations.

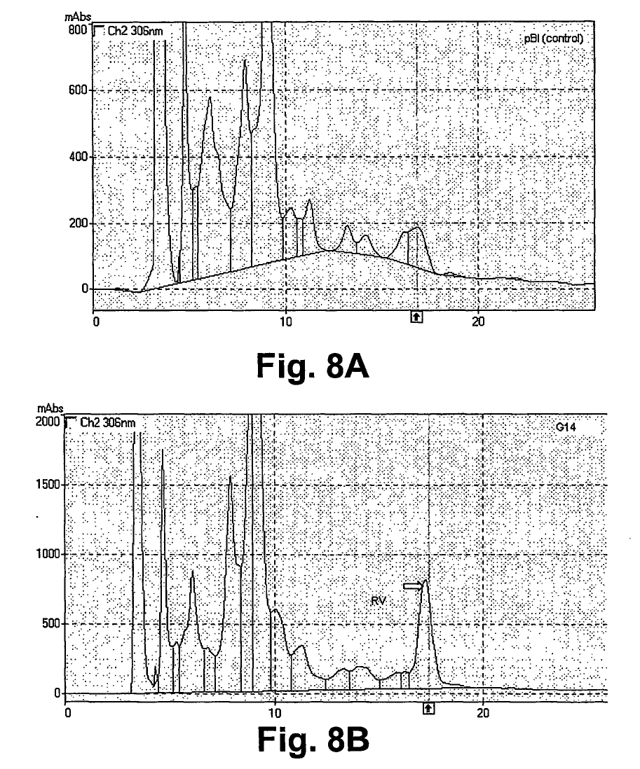

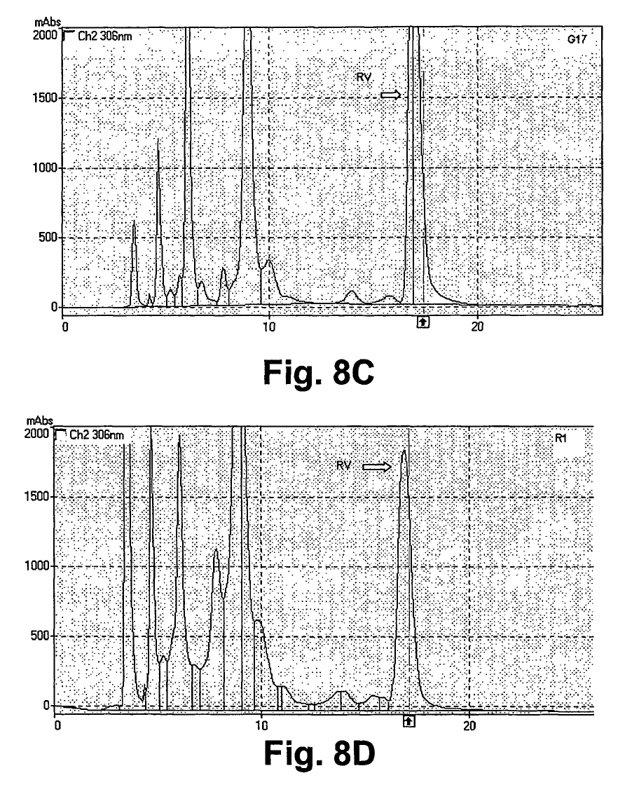

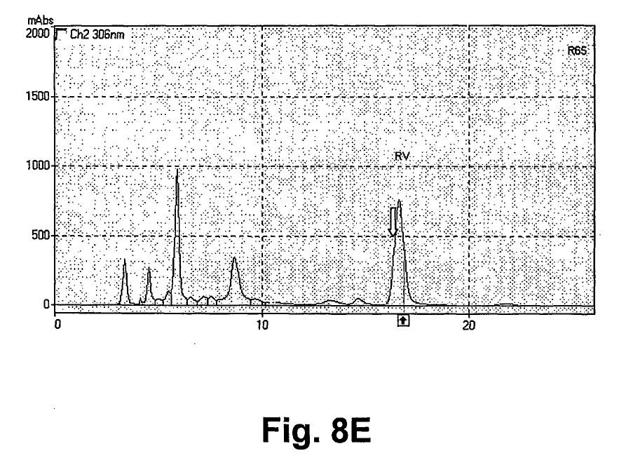

Regeneration from tissue culture of the transgenic plants with >3 ug/g.f.w. of RV expression shows that the transgene is still stable after 10 generation of regeneration in vitro. HPLC analysis of RV of different transgenic plants is shown in FIGS. 8A-E. The method of HPLC analysis is as follows:

Resveratrol Extraction from Putative L. sativa cv Red Salad Bowl and N. tabacum cv Xanthi

Resveratrol was extracted from putative transgenic L. sativa cv Red Salad Bowl and N. tabacum cv Xanthi as described in Hain R. (Plant. Mol. Biol.15:325-335, 1990) and Celotti E. (J. Chromatogr. A.730;47-52, 1996) with some modification.

5 g of fresh leaves from putative transgenic L. sativa cv Red Salad Bowl and N. tabacum cv Xanthi were ground in liquid nitrogen with mortar and pestle until powdery. Before the powder started to thaw, 1 ml/g fresh weight of methanol (MeOH) was added for extraction at room temperature for 24 hours. After MeOH extraction, the slurry was centrifuged at 7,000 rpm for 15 minutes to remove the cell debris. 2 volumes of milli-Q water were added to the supernatant. This solution was mixed with 9 ml ethyl acetate for 15 seconds. The tubes were cooled top 4° C. for 3 minutes, then placed in −20° C. for 5 minutes. The cooling of the tube improved the separation between the organic phase and the water phase. The organic phase was recovered while the aqueous phase was further extracted twice with 6 ml ethyl acetate. The organic phase was recovered and anhydrous sodium sulphate was added to remove any traces of water. The water phase was used for anthocyanin determination. The organic phase was concentrated in Eppendorf™ vacuum concentrator. 50 μl of MeOH were added to the dried samples.

HPLC Analyzes

The putative transgenic samples were analyzed using Shimadzu model CBM-10A reverse-phase HPLC system (Japan). 6 ng/μl of chemically synthesized trans-resveratrol (Sigma, U.S.A.) were used as the standard. 50 μl of the extracted samples were run through C18 column (125 mm×5 mm) with water:glacial acetic acid:acetonitrile (75:5:20) as the mobile phase. The flow rate was set at 0.5 ml/min and diode array UV detector (SPD-M10AVP) was set at 306 nm. The retention time of resveratrol was about 17 minutes.

Anthocyanins Determination Using Visible Light Spectrophotometry

The method used for the determination of anthocyanins in putative transgenic L. sativa cv Red Salad Bowl and N. tabacum cv Xanthi was as described in Mancinelli (1990) with some modification. Extraction method followed that of resveratrol determination because the anthocyanins dissolved into the water phase while the resveratrol dissolved into the organic phase. 1 ml of the water phase was read by Du® 650 spectrophotometer (Beckman, U.S.A.) in a light path 10 mm cuvette. The absorbances at 530 nm and 657 nm were determined and the anthocyanins level was calculated by the formula (A₅₃₀-0.25A₆₅₇)/(fresh weight in gram). The anthocyanins concentration was expressed as A₅₃₀/g. The absorption peak of anthocyanins was measured at absorbance 530 nm. As for the absorption peak at 657 nm, it measured the degraded products of chlorophyll in acidic MeOH.

1. A transformed plant transformed with a stilbene synthase (STS) gene or portion of a STS gene, said STS gene encoding a specific STS enzyme, said specific STS enzyme synthesizing constitutive levels of said specific stilbene in said transformed plant, said transformed plant in the natural untransformed state contains high levels of precursors for said STS gene.

2. A transformed plant according to claim 1, wherein said plant is visually perceived to be red, said STS gene is resveratrol synthase (RS) gene, said specific STS enzyme is RS, and said specific stilbene is resveratrol.

3. A transformed plant according to claim 2, said transformed plant is red-leaf lettuce.

4. A transformed plant according to claim 3, said plant is red-leaf lettuce capable of producing viable seeds.

5. A method of producing a transgenic plant containing a specific transgenic stilbene synthase (STS) enzyme transformed therein, said transgenic plant obtained from a recipient plant comprising:
a) selecting a recipient plant containing high levels of the precursors of the transgenic STS gene;
b) providing a genetic vector comprising an STS gene or a portion of an STS gene encoding said specific STS enzyme, said STS gene the portion of said STS gene being provided with a promoter suitable for constitutive expression of said specific STS enzyme in said recipient plant;
c) transforming said genetic vector into the recipient plant; and
d) selecting and growing said transformed plant containing high and constitute levels of the transgenic stilbene.

6. A method according to claim 5, wherein said selecting step further comprises growing said recipient plant as callus culture in a tissue culture system, and analyzing said callus for the levels of said precursors for said STS enzyme.

7. A method of producing a transgenic plant containing a transgenic reseveratrol synthase (RS) enzyme, said transgenic plant obtained from a recipient plant comprising:
a) selecting a recipient plant that contains high levels of 4-coumaryl-CoA and malonyl-CoA;
b) providing a genetic vector comprising an RS gene or a portion of an RS gene encoding an RS enzyme, said RS gene or the portion of said RS gene being provided with a promoter suitable for constitutive expression of said RS enzyme in said recipient plant;
c) transforming said genetic vector into said recipient plant; and
d) selecting and growing said transformed plant containing high and constitutive levels of transgenic resveratrol.

8. A method of producing a transgenic plant containing a transgenic resveratrol synthase (RS) enzyme, said transgenic plant obtained from a recipient plant comprising:
a) selecting a recipient plant having edible portions containing high and constitutive levels of anthocyanin;
b) providing a genetic vector comprising an RS gene or a portion of an RS gene encoding an RS enzyme, said RS gene or portion of said RS gene being provided with a promoter suitable for constitutive expression of said RS enzyme in said recipient plant;
c) transforming said genetic vector into said recipient plant; and
d) selecting and growing said transformed plant containing high and constitutive levels of resveratrol in said edible portions.

9. A method according to claim 8, wherein recipient plant is red-leaf lettuce.

10. A drink containing unfermented juice from an edible portion of a transformed plant, said juice containing at least 50 μg/ml of resveratrol.

11. Dried fruits and vegetables containing at least 30 μg resveratrol per gram dry weight.

12. Powdered plant extracts containing at least 30 μg resveratrol per gram dry weight.


SEQUENCE LISTING (4 sequences)

SEQ ID NO: 1 - DNA, 1547 bp, Vitis vinifera cv. Red Flame
  G14RS
cttcctcaacttaatcttaggccttaatttgagtacgtagctgggatcaatggcttcagtcgaggaaattagaaacgctcaacgtgccaagggtccggccaccatcctagccattggcacagctacccccgaccactgtgtctaccagtctgattatgctgatttctatttcagggtcactaagagcgagcacatgactgcgttgaagaagaagttcaatcgcatatgtaagtatattcatgcattaattttcttacatgcataacagttctgtatatatgtgactgacactaggtgaggctcacctccaagtgaatgaatgttgcaatctttctagagtatagcttttagataaaatactacagaaaacttgaaaattattttacttcagtagctaatattcatttcatctgacttaaatggcttgaagagctgttctttgaatcatgtcgcattgccagctataattaagaataaccttctatacttgcttcaatgttaaatgcatgtcgatcatcttcaacgatattctatatcacttgttgattggtaaaactaatgtgttcatgttatttcatttacaggtgacaaatccatgaatcaagaagcgttacattcatttgaccgaagaaatgcttgaggagcacccaaacattggtgcttatatggctccatctcttaacatacgccaagagattatcactgccgaggtacccaagctcggtaaggaagcagcactgaaggctcttaaagagtggggtcagcctaaatcgaagatcacccaccttgtattttgtaccacctcaggtgtagaaatgcctggtgcagattataaactcgctaatctcttaggcctcgaaccatctgtcagaagagtgagttgtaccatcaaggggctatgcaggtggaactgtccttcgaaccgctaagggatatgcagagaataatgcaggagcacgaagatacatgggtgtgactctgaggaatcacagttgttacaattgcccgcccttccgaagatgctttggactctttagttggccaagccctttttggtgatgggtctgcagctgtaatcgaggatcagatccggatatctcaattgaacgaccactcttccagcttgttcagcagcccaaacatttatgcctaattctgcaggtgccattgcaggaaacttacgtgaggttgggactcacctttcgtttgtggcccaatgtgcccactttaatttctgagaacatagagaaatgtttgactcaggttttgacccacttggtattagcgattggaactcgttattttggattgctcacccaggtggccctcgaattcttgatgcagttgaagcaaaactcaatttagataaaaagaaactcgaagcaacgaggcatgtgctaagtgagtatggaaacatgtcaagtgcatgtgtgttgtttatttttggatgagaatgagaaagaatcccttaagggggagaggggccaccacggggtgaaggattgattgggagtattattcgtttgaccaggcttgactatgaactg

SEQ ID NO: 2 - DNA, 1648 bp, Vitis vinifera cv. Red Flame
  G17RS
cttcctcaacttaatcttaggccttaatttgagtacgtagctgggatcaatggcttcagtcgaggaaattagaaacgctcaacgtgccaagggtccggccaccatcctagccattggcacagctacccccgaccactgtgtctaccagtctgattatgccgatttctatttcagggtcactaagagcgagcacatgactgcgttgaagaagaagtaagtatattcatgcattaattttcttacatgcataacagtttctgtatatatgtgactgacactaggtgaggctcacctccaagtgaatgaatgttgcaatctttctagagtatagcttttagataaaatactacagaaaacttgaaaattattttacttcagtagctaatattcatttcatctgactgaaatggcttgaagagctgttcttttgaatcatgtcgcattatcagctattcttaagaataacctcctatactcgatcgaaagttaaattcctgtccatcacctctcaacgataggcgataacacttgtggattggtaaaattaccgggacaaatccatgatcaagaagcgttacattcatttgaccgaagaaatgcttgaggagcacccaaacattggtgcttatatggctccatctcttaacatacgccaagagattatcactgccgaggtacccaagctcggtaaggaagcagcattgaaggctcttaaagagtggggtcagcctaaatcgaagatcacccaccttgtattttgtaccacctcaggtgtagaaatgcctggtgcagattataaactcgctaatctcttaggcctcgaaccatcagacagaagagtgatgttgtaccatcaagggtgctatgcgggtggaactgtccttcggagaaatgtttgactcaggcttttgacccacttggtattagcgattggaactcgttattttggattgctcacccaggtggccctgcaattaaccgctaaggatgctgcagagaataatgcaggagcacgagatcttgtggtgtgctctgagatcacagttgttacatttcggggcccttccgaagatgctttggactctttagttggccaagccctttttggtgatgggtctgcagctgtaatcgtaggatcagatccggatatctcaattgaacgaccactcttccagcttgtctcagcagcccaaacatttattcctaattctgcaggtgccattgcaggaaacttacgtgaggtgggactcacctttcatttgtggcccaatgtgcccactttaatttctgagaacatagagaaatgtttgactcaggcttttgacccacttggtattagcgattggaactcgttattttggattgctcacccaggtggccctgcaattcttgatgcagttgaagcaaaactcaatttagataaaaagaaactcgaagcaacgaggcatgtgctaagtgagtatggaaacatgtcaagtgcatgtgtgttgtttattttggatgagatgagaaagaaatcccttgagggggagagggccaccacgggtgaaggattggattggggagtattattcggttttggaccaggcttgactattgaactattgtgttgcatagcattcctatggttacaaattaagtgaaggaa

SEQ ID NO: 3 - DNA, 1289 bp, Vitis vinifera cv. Red Flame
  R1RS
cttcctcaacttaatcttaagcatcaatgtcagaacgtatctggcatccatggcttcagttgaggaatttaggaggcgctcaacgctgccaagggtccggccactatcctagccattgaccactgtagtctaccagtctgattatggctgattactatttcagggtcactaagagccgagcacatgactgagttgaagaagaagttcaatcgcatatatggacaatctcatattacaaatcaatgatcaagaagcgttactattcacttgactcgaagaaatttgccttttcgaggagccacccccaaacctatttggttgccccttattcattggccttcccattccttcctttaaccattaccgcccaagagatttattccaccttgccttgaggtacctagacctttggtagggattgcgccagcatttgaagcggctctaaagagtgggccaaccaaagtccaagatcaccatcttgtattttgtacaacctccggtgtagaaatgccggtgcggattacaaactcgcctaatctcttaggtcttgaaacatccgttagaagggtgatgttgtaccatcaagggtgttatgcaggtggaactgtccttcgaactgctaaggatcttgcagaaaataatgcaggagcacgagttcttgtggtatgctctgagatcactgttgttacattccgtgggccttccgaagatgctttggactctttagttgccctttttggtgatgggtccttcacgctgtgattgttggatccagatcccagatgtctcgattgaacgaccactcttccaacacttgtttcaagccggccccaaacatttattcctaatccagcaggagccattgccggaaacttacgtgaggtggggctcacttttcatttgtggcccaatgcgcctactttgatttctgagaacatagagaaatgtttgacccaggcttttgacccacttggtattagcgattggaactcgttattttggattgctcacccaggtggccctgcaattctcgatgcagttgaagcaaaactcaatttagagaaaaaagaaactgaagcaacaaggcacgtgtaagtgtgtatggtaacatgtctagtgcatgtgtgttgtttattttggatgagatgagaaagaaatccttaaaaggggaaaaagcgaccacaggtgaaggattagattggggatgtactatttggttttgggcctggcttgaccattgaaactgttgtgctgtatagcgttctacggttacaaattaagtgaaggaa

SEQ ID NO: 4 - DNA, 1236 bp, Vitis vinifera cv. Red Flame
  R65RS
rrscttcctcaacttaatcttaagcttcaatttcagtacgtatctggcatccatggcttcagtcgaggaatttagaaacgctcaacgtgccaagggtccggccaccatcctagccattgcgacagctacccccgaccactgtgtctaccagtctgattatgctgattactatttcagggtcactaagagcgagcacatgactgagttgaagaagaagttcaatcgcatatgtgacaaatccatgatcagaggtacattcacttgcccgaagaaatgcttgaggagcacccaaacattggtgcttatatggctccatctcttaacatacgccaagagattattactgctgaggtacctagacttggtagggatgcagcattgaaggctcttaaagagtggggccaaccaaagtccaagatcacccatcttgtattttgtacaacctccggtgtagaaatgcccggtgcggattacaaacttgctaatctcttaggtcttgaaacatcggttagaagggtgatgttgtaccatcaagggtgctatgcaggtggaactgtccttcgaaccgctaaggatcttgcagaaaataatgcaggagcacgagttcttgtggtgtgctctgagatcactgttgttacattccgtgggccttccgaagatgctttggactctttagttggccaagccctttttggtgatgggtcttcagctgtgattgttggatcagatccagatgtctcgattgaacgaccactcttccaacttgtttcagcagcccaaacatttattcctaattcagcaggagccattgccggaaacttacgtgaggtggggctcacctttcatttgtggcccaatgtgcctactttgatttctgagaacatagagaaatgcttgtcccaggcttttgacccacttggtattagcgattggttctcattattttggattgctcacccaggtggccctgcaattctcgatgcagttgaagcaaaactcaatttagagaaaaagaaacttgaagcaacaaggcacgtgttaagtgagtacggtaacatgtcaagtgcatgtgtgttgtttgttttggatgagatgagaaagaaatccctaaagggggaaaaagccaccactggtgaaggattggattggggagtactatttggttttgggccaggcttgaccatcgaaactgttgtgctacatagcattcctacggttacaaattaagtgaaggaa